(19) United States
(12) Patent Application Publication
(10) Pub. No.: US 2017/0304164 A1
(43) Pub. Date: Oct. 26, 2017

(54) COMPOSITIONS, APPARATUS AND METHODS FOR MONITORING AND IMPROVING ORAL HEALTH

(71) Applicant: Berkeley NOX Limited, Galway (IE)

(72) Inventor: Shawn J. Green, Sacramento, CA (US)

(21) Appl. No.: 15/492,925

(22) Filed: Apr. 20, 2017

Related U.S. Application Data
(60) Provisional application No. 62/325,635, filed on Apr. 21, 2016.

(51) Int. Cl. A61K 8/19; G01N 21/78; A61K 8/02; A61K 8/21; G01N 33/84; A61B 5/145; A61K 8/67; A61Q 11/00; A61B 10/00; A61K 8/368; G01N 21/77; G01N 21/75

(52) U.S. Cl. CPC A61K 8/19; A61B 10/0051; G01N 21/78; A61K 8/0216; A61K 8/21; A61K 8/368; G01N 33/84; A61B 5/14507; A61K 8/676; A61Q 11/00; A61B 2562/0295; A61K 2800/92; G01N 2021/752; G01N 2021/7759; A61K 2800/88

(57) ABSTRACT

The present disclosure relates to methods, compositions and apparatus for monitoring and improving oral health by utilizing information about nitric oxide levels in an individual's oral cavity. In an embodiment, the disclosure provides methods and apparatus for monitoring nitric oxide metabolites in saliva; this information may be utilized as it relates to improving oral health, i.e. monitoring and improving oral hygiene and increasing the consumption of nitric oxide-potent foods. In an embodiment, methods for improving oral health in a subject comprising, use of an oral cleanser by an individual, wherein the oral cleanser comprises a potassium nitrate source; and use of a saliva test strip for measuring nitrite and nitrogen oxides in the oral cavity of the individual are provided. In an embodiment, a novel nitrate-rich gum composition is provided.

### TECHNICAL FIELD

Embodiments of the disclosure relate generally to compositions, methods and apparatus for monitoring oral health. In an embodiment, the compositions, methods and apparatus are particularly suited for providing information about nitric oxide levels in an individual's oral cavity. In an embodiment, the present invention provides methods and apparatus for monitoring nitric oxide metabolites in saliva; this information may be utilized as it relates to improving oral health, i.e. monitoring and improving oral hygiene and increasing the consumption of nitric oxide-rich foods.

### BACKGROUND OF THE INVENTION

Like many areas of the body, the mouth is teeming with bacteria, most of them harmless. Normally the body's natural defenses and good oral health care, such as daily brushing and flossing, can keep these bacteria under control. However, without proper oral hygiene, bacteria can reach levels that might lead to oral infections, as well as tooth decay and gum disease. In addition, certain medications, such as decongestants, antihistamines, painkillers and diuretics, can reduce saliva flow. Saliva is important because it washes away food and neutralizes acids produced by bacteria in the mouth, helping to protect the mouth from microbial invasion or overgrowth that might lead to disease.

Oral health is essential to general health and quality of life. It is a state of being free from mouth and facial pain, oral and throat cancer, oral infection and sores, periodontal (gum) disease, tooth decay, tooth loss, and other diseases and disorders that limit an individual's capacity in biting, chewing, smiling, speaking, and psychosocial wellbeing. The most common oral diseases are dental cavities, periodontal (gum) disease, oral cancer, oral infectious diseases, trauma from injuries, and hereditary lesions.

Dental cavities are widely recognized as constituting serious concern with regard to oral health. Worldwide, 60-90% of school children and nearly 100% of adults have dental cavities, often leading to pain and discomfort. Tooth decay (cavities) is one of the most common chronic conditions of childhood in the United States. Untreated tooth decay can cause pain and infections that may lead to problems with eating, speaking, playing, and learning. In the United States, about 1 of 5 (20%) children aged 5 to 11 years have at least one untreated decayed tooth; and about 1 of 7 (13%) adolescents aged 12 to 19 years have at least one untreated decayed tooth. The percentage of children and adolescents aged 5 to 19 years with untreated tooth decay is twice as high for those from low-income families (25%) compared with children from higher-income households (11%). There are threats to oral health across the lifespan. Nearly one-third of all adults in the United States have untreated tooth decay.

Periodontitis is a set of inflammatory diseases affecting the periodontium, i.e., the tissues that surround and support the teeth. Periodontitis involves progressive loss of the alveolar bone around the teeth, and, if left untreated, can lead to the loosening and subsequent loss of teeth. Periodontitis is caused by microorganisms that adhere to and grow on the tooth's surfaces, along with an overly aggressive immune response against these microorganisms. Periodontitis manifests as painful, red, swollen gums, with abundant plaque. Symptoms may include redness or bleeding of gums while brushing teeth, using dental floss, or biting into hard food (e.g. apples); recurrent swelling of the gum; halitosis and a persistent metallic taste in the mouth; gingival recession resulting in apparent lengthening of teeth; deep pockets between the teeth and the gums (pockets are sites where the attachment has been gradually destroyed by collagenases); and loose teeth. Periodontitis also has been shown to have effects outside of the mouth. For example, periodontitis has been linked to increased inflammation as indicated by increased levels of C-reactive protein and Interleukin-6. In addition, periodontitis has been shown to increase the risk for a number of other diseases, including but not limited to, stroke, myocardial infarction, atherosclerosis, diabetes, and pre-term labor. Severe periodontal (gum) disease, which may result in tooth loss, is found in 15-20% of middle-aged (35-44 years) adults. In addition, dental cavities and periodontal disease are major causes of tooth loss. Complete loss of natural teeth is widespread and particularly affects older people. Globally, about 30% of people aged 65-74 have no natural teeth.

Oral cancer constitutes another concern in oral health. The incidence of oral cancer ranges from one to 10 cases per 100,000 people in most countries. The prevalence of oral cancer is relatively higher in men, in older people, and among people of low education and low income. Tobacco and alcohol are major causal factors.

Oral health might affect, be affected by, or contribute to various diseases and conditions, including: endocarditis, an infection of the inner lining of the heart (endocardium), and typically occurs when bacteria or other germs from another part of the body, such as the mouth, spread through the bloodstream and attach to damaged areas in the heart; cardiovascular disease, research suggests that heart disease, clogged arteries and stroke might be linked to the inflammation and infections that oral bacteria can cause; pregnancy and birth, periodontitis has been linked to premature birth and low birth weight; diabetes, since diabetes reduces the body's resistance to infection the gums are put to at risk and gum disease appears to be more frequent and severe among people who have diabetes (research shows that people who have gum disease have a harder time controlling their blood sugar levels); HIV/AIDS, oral problems, such as painful mucosal lesions, are common in people who have HIV/AIDS in addition, almost half (40-50%) of people who are HIV-positive have oral fungal, bacterial or viral infections; osteoporosis, since this condition causes bones to become weak and brittle it might be linked with periodontal bone loss and tooth loss; Alzheimer's disease, tooth loss before age 35 might be a risk factor for Alzheimer's disease; other conditions that might be linked to oral health include Sjogren's syndrome an immune system disorder that causes dry mouth and eating disorders.

The burden of oral diseases and other chronic diseases can be decreased simultaneously by addressing common risk factors. These include: decreasing sugar intake and maintaining a well-balanced nutritional intake to prevent tooth decay and premature tooth loss; consuming fruit and vegetables that can protect against oral cancer; stopping tobacco use and decreasing alcohol consumption to reduce the risk of oral cancers, periodontal disease and tooth loss; using protective sports and motor vehicle equipment to reduce the risk of facial injuries; safe physical environments, and most importantly, ensuring proper oral hygiene. Dental cavities can be prevented by maintaining a constant low level of fluoride in the oral cavity. Fluoride can be obtained from fluoridated drinking water, salt, milk and toothpaste, as well as from professionally-applied fluoride or mouth rinse. Long-term exposure to an optimal level of fluoride results in fewer dental cavities in both children and adults. As a general rule, it is recommended that all individuals practice good oral hygiene every day by brushing their teeth at least twice a day, by flossing daily, eating a healthy diet and limiting between-meal snacks, replacing toothbrushes every three to four months and scheduling regular dental checkups.

### Salivary Nitrate and Oral Health

While numerous diagnoses and physical assessments require the use of sophisticated equipment and extensive testing by specialists in clinics, a deeper understanding of biomarkers and their significance may provide an opportunity to utilize this information in less obtrusive, less expensive, and less burdensome way. Recent technological advances, along with a better comprehension of metabolic, biochemical and physiological processes have enabled individuals to assume greater responsibility for their own wellness, health and physical fitness. Detection of such biomarkers may provide information that can assist individuals in assessing physiological status, and consequently making appropriate adjustments.

Numerous commercial tests are currently available to the general public, and such tests enable consumers to monitor their health in the privacy of their own homes, without the inconvenience or time-consuming activity of travelling to a healthcare or laboratory facility. Examples of such tests include the pregnancy test (biomarker detected is human chorionic gonadotropin (HCG), test media is urine), blood glucose test (biomarker detected is glucose metabolite, test media is blood), cholesterol test (biomarkers detected include HDL, LDL, and triglycerides, test media is blood), and prostate specific antigen (PSA) test (biomarker detected is PSA, test media is blood). The rising popularity of such tests supports the notion that consumers are becoming increasingly proactive about monitoring various aspects of their health, presumably in an effort to prevent illness and improve quality of life.

One set of biomarkers of interest with regard to oral hygiene comprises nitrite and nitrogen oxide. Nitrite, derived from nitrate through nitrate reducing bacteria on the tongue surface, is considered cytocidal and cytostatic to common oral pathogens involved in caries and in periodontal disease. Therefore, an increase in nitrate secretion and a subsequent increase in salivary nitrite may contribute to the overall protective effect against those infections conditions, affecting both hard and soft oral tissues. It is known that salivary glands may respond to periodontitis by enhancing the protective potential of saliva. Thus, it is likely that the increment in salivary nitrate-nitrite concentration, in patients with periodontal disease, may be due to an increase in nitrate secretion as a response of salivary glands to the inflammatory process. In accordance with this hypothesis, it has been reported that patients with oral candidiasis have increased salivary nitrates and nitrites concentration.

High salivary nitrate and high nitrate-reducing capacity in the oral cavity was found to be protective against dental caries in a study of 209 children. Salivary nitrate and nitrite levels, counts of Streptococcus mutans and Lactobacillus spp., and caries experience were recorded, and compared with control subjects, a significant reduction in caries and counts of Streptococcus mutans and Lactobacillus spp. was found in patients with high salivary nitrate. Production of nitrite from salivary nitrate by commensal nitrate-reducing bacteria likely limit the growth of cariogenic bacteria as a result of the production of antimicrobial oxides of nitrogen, including nitric oxide. (J. J. Doel, M. P. Hector, C. V. Amirtham, L. A. Al-Anzan, N. Benjamin, R. P. Allaker, Protective effect of salivary nitrate and microbial reductase activity against caries, Eur. J. Oral Sci. 112 (2004) 424-428.)

Others have shown that the antimicrobial agent nitric oxide is formed in the mouth and its concentration is directly related to salivary nitrite, which in turn is related in part to dietary nitrate intake. Here the investigators showed that nitrite, via the bioconversion from nitrate, under acidic conditions had an inhibitory effect, through NO production, on Streptococcus mutans, Lactobacillus casei and Actinomyces naeslundii. Whereas the growth of S. mutans was inhibited by a more acid pH, the addition of nitrite caused a marked, further dose-dependent reduction in bacterial numbers after 24 hours of exposure Similar effects were observed with A. naeslundii and L. casei. The ability of these bacteria to recover from nitrite exposure was also markedly affected by nitrite concentration. At acidity levels below pH 7, low concentrations of nitrite (0.2 mM) caused effective complete killing of S. mutans, with similar effects on the other organisms tested. These results demonstrate that nitrite in saliva blocks cariogenic bacteria. (L. S. Silva Mendez, R. P. Allaker, J. M. Hardie, N. Benjamin, Antimicrobial effect of acidified nitrite on cariogenic bacteria, Oral Microbiol. Immunol. 14 (1999) 391-392, C. E. Radcliffe, R. Lamb, A. S. Blinkhorn, D. B. Drucker, Effect of sodium nitrite and ascorbic acid on the growth and acid production of Streptococcus mutans, J. Dent. 31 (2003) 367-370.)

Consistent with other reports, nitrite derived from nitrate under acidic conditions inhibited the growth of periodontal disease pathogens Fusobacterium nucleatum, Eikenella corrodens and Porphyromonas gingivalis. (P. Allaker, L. S. Silva Mendez, J. M. Hardie, N. Benjamin, Antimicrobial effect of acidified nitrite on periodontal bacteria, Oral Microbiol. Immunol. 16 (2001) 253-256.) Sanchez et al (2014) reports that an increase in nitrate likely contributes to the overall protective effect against periodontal-associated pathogens affecting both hard and soft oral tissues. They suggest that an increase in salivary nitrate-nitrite concentration, in patients with periodontal disease, is due to an increase in nitrate secretion as a response of salivary glands to the inflammatory process. In accordance with this hypothesis, it has been reported that patients with oral candidiasis have increased salivary nitrates and nitrites concentration. In both case, it is suggested that the elevation of nitrate in the oral cavity during advance disease is an immune response to such infection. (Total salivary nitrates and nitrites in oral health and periodontal disease. Sanchez G A, Miozza V A, Delgado A, Busch L. Nitric Oxide. 2014 Jan. 30; 36:31-5.). Numerous additional studies have highlighted the benefits of salivary nitrate levels: Li et al (2007) suggest that elevated salivary nitrate reduces oral acidity hence protects against tooth decay. (Oral Microbiol Immunol. 2007 February; 22(1):67-71. Salivary nitrate—an ecological factor in reducing oral acidity. Li H1, Thompson I, Carter P, Whiteley A, Bailey M, Leifert C, Killham K.) Along similar lines, Radcliffe (2002) suggests that exogenous nitrite acidified by metabolic products of acidogenic bacteria in the mouth will be converted to products which inhibit growth of Streptococcus mutans. (Effects of nitrite and nitrate on the growth and acidogenicity of Streptococcus mutans. Radcliffe C E, Akram N C, Hurrell F, Drucker D B. J Dent. 2002 September-November; 30(7-8):325-31) In addition to susceptible acidified nitrite derived from nitrate, including cariogenic bacteria Streptococcus mutans and various periodontal bacteria, Fusobacterium and P. gingivalis, the sulfate-reducing bacteria, Desulfovibrio spp., which is considered an etiologic agent associated with chronic periodontitis and implicated to sulfate odors is directly inhibited by both nitrite and nitrate. Here, growth is inhibited with 0.2 mM nitrate which is consistent with other observations. Salivary nitrate and nitrite may have antimicrobial effects on Desulfovibrio species. (Mitsui T, Fujihara M, Harasawa R. Biosci Biotechnol Biochem. 2013; 77(12):2489)

As mentioned previously, a number of devices for collecting and testing bodily fluids (i.e., saliva) for the presence of various metabolites exist in the art. In the context of providing a relatively quick and inexpensive sample collecting device and associated testing system, there exist several approaches for collecting a sample fluid, expressing the sample fluid in a test device and performing an assay of the sample. Examples of these types of testing systems include U.S. Pat. Nos. 5,965,453; 6,027,943; 4,895,808; 4,943,522; 6,267,722, 5,393,496, 7,763,433 and 7,507,374.

Prior art devices typically include a sample collector, a container for holding the sample collector and a testing apparatus. One type of sample collector typically includes an absorbent pad for absorbing the target fluid and a holder for holding the sample as the sample is being collected. The sample is then transferred to a sample container or test device by using one of a variety of known approaches including a mechanism for expressing the sample into a sample container (see U.S. Pat. No. 5,268,148) or dipping the sample collector into a test solution (see U.S. Pat. No. 4,895,808) or using a second filter or absorbent pad to transfer the fluid from the collector pad to an intermediate container or test device. Sample collectors may also include a sponge or chemical reagent disposed on a filter strip, which may indicate that an adequate sample is collected (see U.S. Pat. No. 5,393,496).

Strip tests with an absorption pad or chemical reagent test pad are often used in applications for home testing and rapid point of care testing. In some instances, the chemical reagent test pad also serves as the absorption pad.

A major limitation of using the above-mentioned tests for saliva is that since these tests typically incorporate at least one or more chemicals into the test pad/strip, they may not be directly- or safely-inserted into the mouth: in measuring metabolites found in the mouth, the chemical reagent test pad cannot be directly inserted into the mouth, because the chemical reagents can be harmful or toxic. Hence, in the case of saliva, a separated collection system is always necessary and is usually a separate device or apparatus or approach to transfer fluid to the test pad. An example of such a device is the Neogenis indicator strip, which actually uses one's hand or finger to transfer the saliva to the test strip, i.e., spit on the finger and transfer spit to chemical test pad. Inevitably, such a design, where the saliva must first be obtained from the mouth and then transferred (i.e. by a finger) to the test pad, is not only awkward, but can also potentially contaminate results.

What is needed is a novel approach to oral hygiene whereby information concerning the levels of salivary nitric oxide, and analytes thereof may be utilized in such a way as to maximize the antimicrobial activity of such analytes and consequently improve oral health. More particularly, what is needed is a system utilizing a test that may be used directly in the oral cavity for monitoring nitric oxide status wherein such information is then incorporated into a methodology for improving oral health.

### SUMMARY OF THE INVENTION

The present invention is related to methods and devices for the monitoring and improving oral hygiene. In one embodiment, the present disclosure provides a unique regimen comprising the use of nitrate donor, such as a nitrate-rich gum or oral cleanser having a potassium nitrate ion source used in conjunction with a saliva strip that is a single device used to collect, transfer, and measure saliva nitric oxide analytes, including, but not limited, to nitrite, a precursor and biomarker for nitric oxide, and methods for correlating such measurements to optimize oral health.

Through the detection of saliva analyte and a biomarker of nitric oxide, the present disclosure enables individuals to make real-time adjustments to oral hygiene regimens and to optimize oral health. According to the present disclosure users are able to rapidly, in a real-time fashion, evaluate nitric oxide levels in their oral cavities, and determine a corrective course for improving and maintaining oral hygiene.

The present disclosure provides a novel programmatic approach to oral hygiene comprising the use of a nitrate donor such as a nitrate rich food type substance (i.e. gum) or oral cleaners (including but not limited to toothpaste, mouth wash, dental floss), the use of saliva test strips to monitor nitrogen oxides levels generated in the mouth, and an optional compliance component that receives data concerning the oral cavity and triggers a prompt for the user to take corrective action as necessary. The oral cleaners may comprise a storage stable, anti-plaque, anti-gingivitis, and anti-hypersensitive toothpaste comprising an orally acceptable dental vehicle containing a nitric oxide precursor(s), including but not limited to potassium nitrate ions. Additional oral cleaners may further include mouthwash containing appropriate nitrite reducing agents that effectively provide anti-caries activity to reduce dental plaque and gingivitis in humans. Oral cleaners apparent to those skilled in the art, including dental floss, are included within the scope of the present invention. Saliva test strips utilized in the present invention may include strips as described in U.S. Pat. No. 9,360,490, wherein such strips comprise an elongated colorimetric test strip, wherein the strip contains a scored mark at the midpoint of the strip and in certain embodiments, one end of the strip contains a first absorbent pad, and the opposite end contains a second absorbent pad at the opposite end: the first absorbent pad comprises a fluid collection pad, and the second absorbent pad comprises a test pad. The present invention comprises the use of a nitric oxide generating paste and mouthwash used in context with intra-daily self-monitoring with a saliva test strip for nitrogen oxide for promoting consumer compliance behavior. Colorimetric test strip outcomes may be recorded and tracked on electronic devices such as wearables or mobile phones providing reminders and updates to both user and dentist or healthcare provider through wireless messaging capabilities as to compliance and adherence to oral healthcare.

Through the use of nitrate-rich food type components, such as gum, comprising a potassium nitrate ion source and easy to use saliva test devices to collect and measure nitric oxide analytes in saliva fluid, it is an aspect of the present disclosure to promote healthy oral hygiene habits.

Through the use of oral cleansers comprising a potassium nitrate ion source and easy to use saliva test devices to collect and measure nitric oxide analytes in saliva fluid, it is an aspect of the present disclosure to promote healthy oral hygiene habits.

Another aspect of the present disclosure is to provide a systematic approach to improving oral health, and thereby decrease the incidence of health issues related to oral health, including but not limited to dental cavities, tooth decay, gum disease, periodontitis, oral cancer.

A further aspect of the present disclosure is to incorporate the use of a rapid, inexpensive, self-administered saliva test device and method to collect and measure nitric oxide analytes in saliva fluids to monitor nitric oxide levels in the oral cavity and to utilize such measurements to improve oral health through the use of antimicrobial toothpaste and mouthwash.

These and other aspects, features and advantages of the present disclosure will become apparent after a review of the following detailed description of the disclosed embodiments and the appended claims.

### BRIEF DESCRIPTION OF THE FIGURES

FIG. 1 provides a graph showing the correlation of saliva nitric oxide status with oral health status.

FIG. 2 provides a graphic depiction of nitric oxide pathways.

FIG. 3 provides a graphic depiction of nitric oxide production in the oral cavity (Adapted from Hezel M P, Weitzberg E, 2015, Oral Disease: 7-16).

FIG. 4 provides a table comparing activities of Listerine®, Chlorhexidine and nitric oxide for anti-plaque therapy.

FIG. 5 provides a graph showing the correlation of nitric oxide status in the oral cavity and self-monitoring for antimicrobial bioactivity.

FIG. 6 provides a graphic depiction of a programmatic approach to oral healthcare.

FIG. 7 provides a graphic depiction of an electronic component of the programmatic approach to oral healthcare described herein.

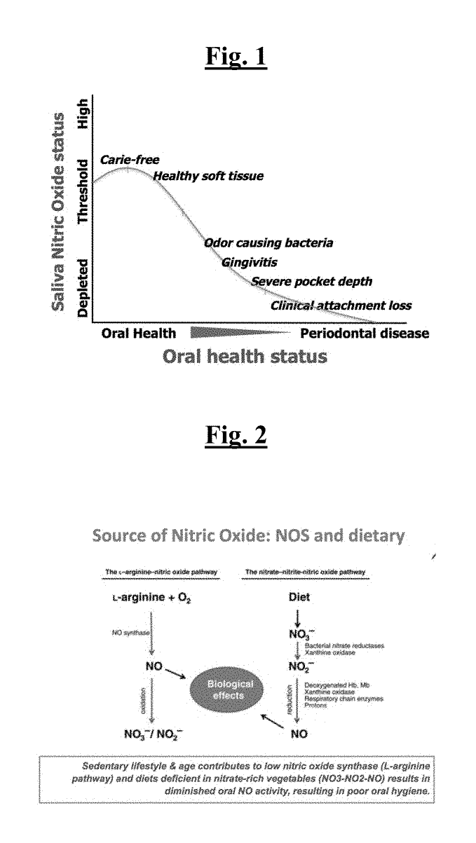

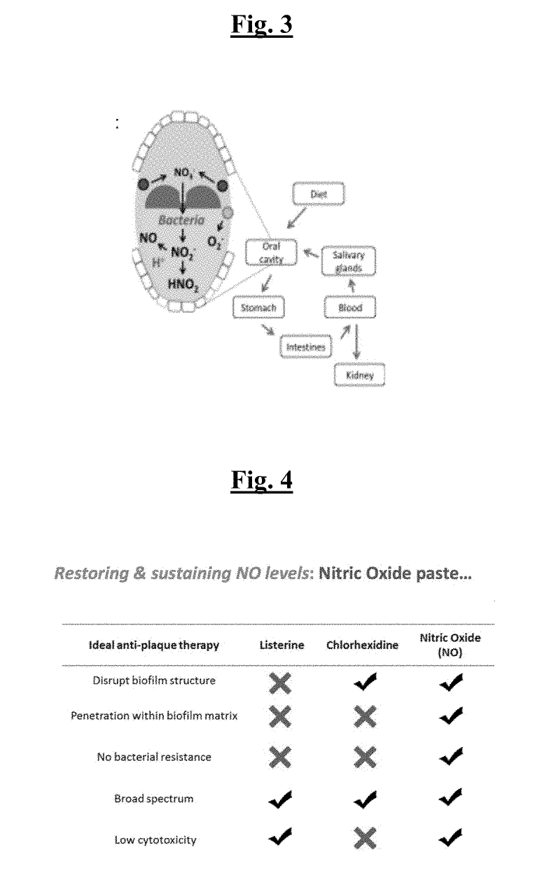

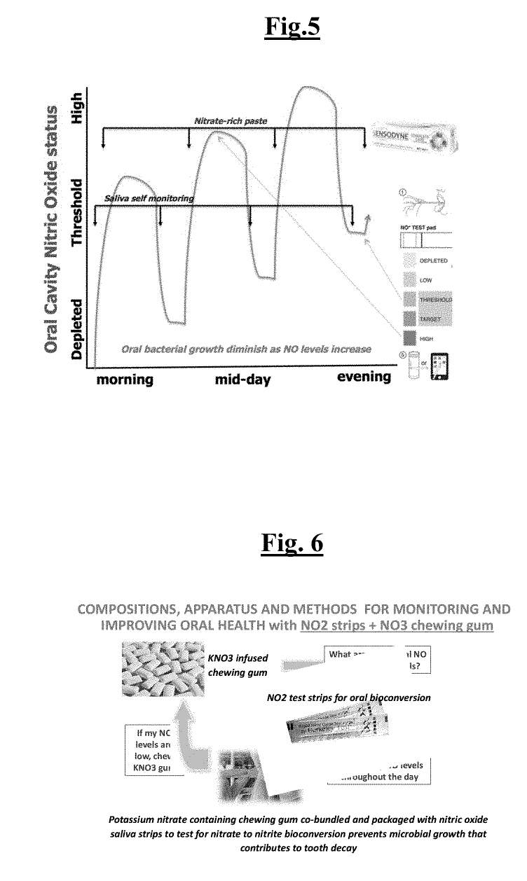

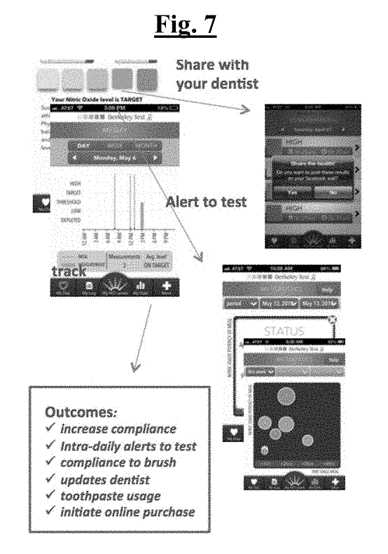

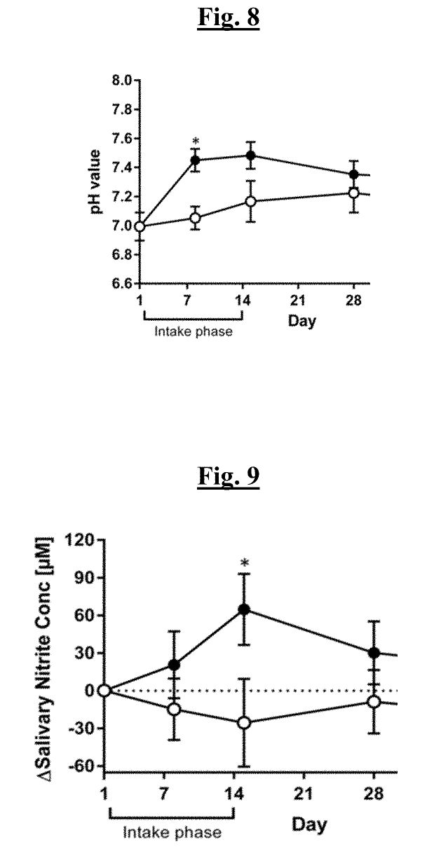

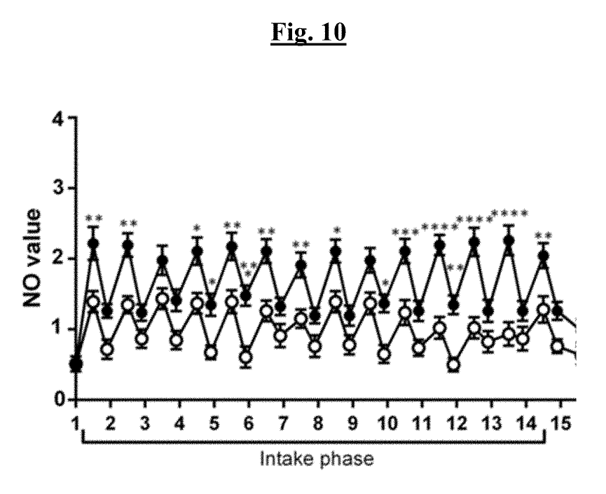

### DETAILED DESCRIPTION OF THE INVENTION

The present invention may be understood more readily by reference to the following detailed description of the specific embodiments included herein. Reference is made to the accompanying drawings, which form a part hereof, and in which is shown, by way of illustration, various embodiments of the present disclosure. Although the present invention has been described with reference to specific details of certain embodiments thereof, it is not intended that such details should be regarded as limitations upon the scope of the invention.

The entire text of the references mentioned herein are hereby incorporated in their entireties by reference including U.S. Provisional Patent Application Ser. No. 62/325,635 filed on Apr. 21, 2016 U.S. Provisional Patent Application Ser. No. 61/697,462 filed on Sep. 6, 2012, U.S. Provisional Patent Application Ser. No. 61/451,221 filed on Mar. 10, 2011 and U.S. Pat. No. 9,360,490.

From detecting cancer, to monitoring blood glucose levels and detecting HCG in order to confirm pregnancy, biomarkers have rapidly gained importance as indicators of physiological health. With advances in the identification of specific biomarkers and their role in indicating various physiological or pathological states, there is heightened interest in incorporating the detection of such biomarkers into commercially available, over-the-counter test kits in order to provide consumers with a convenient and cost-effective option for monitoring and maintaining their physical well-being. Such tests have the potential to analyze bodily fluids, including but not limited to, saliva, sputum, tears, sweat, mucus, serum, semen, urine and blood, to detect biomarkers, including but not limited to, analytes, metabolites, chemicals, hormones, toxins, enzymes, immunoglobulins, proteins, and nucleic acids. Saliva, for example, is known to contain biomarkers including but not limited to nitric oxide analytes and metabolites, uric acid, heavy metals (e.g., lead), hormones (e.g., cortisol, dehydroxyepiandrosterone (DHEA)), toxins and their metabolites (e.g., cotinine), enzymes (e.g., lysozyme, α□amylase), immunoglobulins (e.g., IgA), other proteins (e.g., eosinophil cationic protein) and DNA.

In 1998, the Nobel Prize for medicine was awarded to Robert F. Furchgott, Louis J. Ignarro and Ferid Murad for discovering the importance of nitric oxide in the cardiovascular system. These scientists demonstrated that nitric oxide, is a short-lived, endogenously produced gas that acts as a signaling molecule in the body. Signal transmission by a gas, produced by one cell, which penetrates membranes and regulates the function of other cells was recognized for the first time as an entirely new principle for signaling in the human organism. Related research proved the crucial role that nitric oxide plays in such fundamental biological processes as regulation of blood pressure, functioning and malfunctioning of the immune system, and activation of mechanisms in the central nervous system affecting everything from gastric motility to memory to behavior.

The present disclosure is based upon the discovery that nitric oxide has antimicrobial activity in the oral cavity: more specifically, the inventors herein have observed that oxides of nitrogen produced non-enzymatically and enzymatically from the serial chemical reduction of nitrate to nitrogen oxides, in particular nitric oxide, are potently antimicrobial. In order to optimize and direct the antimicrobial activity of nitric oxide, the inventors have recognized the need to create, sustain and monitor a threshold level of the appropriate precursor within the oral cavity and, specifically, within the proximity of the etiologic pathogens, such as within the subgingival areas or sulcus where bacteria contribute to the advancement of gingivitis and periodontal disease. This disclosure provides a teaching for methods, compositions and devices that enable the optimization of nitrate metabolites to maintain a healthy and antimicrobial environment in the oral cavity of a subject. This goal may be accomplished by the use of nitrate-based food substances (such as gum, lozenges and the like), and/or use of a nitrate-based oral cleansers (such as toothpaste) as antimicrobial agents, optionally combined with saliva tests to promote compliance with oral hygiene regimens.

Nitrate itself is an innocuous precursor, which only produces antimicrobial species when converted to nitrite, and subjected to acid conditions. Lactobacilli sp. transiently produces sufficient acid in the mouth after a carbohydrate meal to control the growth of oral pathogens but a moderate intake of nitrate is a desirable prerequisite in any contaminated environment.

In addition to having antimicrobial activity, nitrite and nitrogen oxide in the oral cavity also has an antiviral effect. Though not wishing to be bound by the following theory, the inventors herein have found that viruses, as opposed to bacteria for example, react differently to a nitrogen oxide complex: namely, when exposed to a nitrogen oxide complex, comprising for example and/or nitrate and acidified nitrite, while the complex may affect replication to a degree, more importantly it modifies the virally infected cells such that the immune system can better recognize the viral particles.

The present disclosure relates to nitrate and/or nitrite enriched food substances (such as gum, lozenges and the like), oral cleansers including but not limited to, toothpaste and mouthwashes as antimicrobial agents for oral hygiene, and to a complex of nitrogen oxides arising from the interaction of nitrite and acid found in sulcus as an antibacterial and antimicrobial composition for the prevention and treatment of caries. Such nitrogen oxides include, but are not limited to nitrate, nitrite and, in particular, nitric oxide. Antimicrobial nitrate/nitrite may be substituted with nitric oxide donor generators, including but not limited to S-nitrosothiol, diazeniumdiolate, NONOate, furoxan, nitroaspirin, organic nitrate.

An active entero-salivary circulation in mammals provides a continuous flow of nitrate into the mouth where it is rapidly reduced to nitrite by bacteria on the tongue. The effect of salivary nitrate excretion is to provide a precursor for the generation of nitrogen oxides by the chemical reduction of the nitrite. In the mouth bacteria rapidly reduce nitrates to nitrites. Nitrite is further reduced in the sulcus to antimicrobial nitrogen oxide species, including nitric oxide. The acid conditions of the sulcus are further elevated and protonate nitrite to form nitrous acid. The nitrous acid in turn dissociates to form oxides of nitrogen as shown below.

NO₂—+H+=HNO₂ 1.

2HNO₂=H₂O+N₂O₃ 2.

N₂O₃=NO+NO₂ 3.

N₂O₃+C₂H₈O₆=2NO+H₂O+C₆H₆O₆ 4.

Endogenous and dietary nitrate is actively concentrated by salivary glands to more than ten times the concentration in plasma and secreted in saliva. Thus, saliva can provide a continuous source of nitrate, assuming nitrate-rich diets are regularly consumed and the endogenous pathway for nitric oxide synthases is intact. Unfortunately, with age and other sedentary conditions, the endogenous pathway is less active. And the levels of dietary nitrate are further diminished if leafy green foods, a rich source of nitrate, are not incorporated into the diet. The present invention provides sources of nitrate based components such as food substances (i.e. gum, lozenges and the like), nitrate based oral cleansers (such as toothpaste and mouthwash) to provide an immediate precursor for the serial reduction in the mouth for antimicrobial activity. In certain embodiments, antimicrobial effects are pronounced if periodontal disease with acid producing bacterial in the sulcus is present.

Oral conversion of nitrate to nitrite is rapid and generally takes place on the surface of the tongue in mammals by commensal nitrate-reducing bacteria (mainly to the posterior third of the tongue) and can be subsequently reduced to nitrogen oxide, including nitric oxide, in the sulcus of infected pockets in the presence of low pH or acidic environments. Alternatively, as disclosed herein, nitrate-based foods, paste or washes in the presence of salicylic acid and/or ascorbic acid can further accelerate the chemical reduction or enhance the formation of antimicrobial nitric oxide in the oral cavity. In an embodiment of the present invention, a moderate acid paste or wash is provided in certain advanced forms of periodontal disease. Provided herein is a novel oral hygiene regimen comprising the use of a source of nitrate for serial reduction in the mouth for the direct treatment and/or prevention of oral disease and oral decay by introduction via food substance, toothpaste and/or mouthwash, coupled with the monitoring of oral nitric oxide level via nitrite/nitrate levels as a biomarker for antimicrobial activity with the intent to increase adherence to daily oral health practice, i.e., brushing teeth (optionally with a nitrate-based tooth paste).

The present disclosure meets the unmet medical and health needs of improving and monitoring oral hygiene. To reduce the number of caries-producing organisms in dental plaque and prevent the advancement of gingivitis and periodontal disease, the present disclosure provides a novel programmatic approach comprising the use of a nitrate-based components, such as food (gum, lozenges and the like) and/or oral cleansers (toothpaste and/or mouthwash) in conjunction with a saliva test strip to monitor the presence of nitrogen oxides after brushing. The saliva test strips may further serve as a reminder to brush repeatedly throughout the day to sustain threshold levels of nitrogen oxide in the oral cavity. Additionally, this programmatic approach extends beyond anti-caries and can be used to treat and prevent infection with C. albicans or other harmful organisms of the oral cavity that are susceptible to nitrogen oxides.

The present disclosure provides a novel source of nitrate in the form of a nitrate rich gum. In an embodiment, the nitrate-rich gum as described herein may be composed of potassium nitrate or a plant powder source derived from spinach, kale, arugula, celery, beets among other leaf greens that are rich in nitrate. The nitrate salt or plant derived nitrate powder source may be mixed with a gum base, which can be, but not limited to, a natural sources, such as chicle, and non-natural sources, including, but not limited to, butadiene-styrene rubber, paraffin, and various waxes used in making chewing gum. In certain embodiments, the gum may further comprise sugar-free sweeteners, such as xylitol, with the addition of both natural and artificial flavors, including spearmint, orange, among other herb and plant sources, such as fennel, and fruit flavors.

The present disclosure provides an approach to oral hygiene comprising a storage stable, anti-plaque, anti-gingivitis, and anti-hypersensitive toothpaste comprising an orally acceptable dental vehicle containing a nitric oxide precursor(s), including but not limited to potassium nitrate ions. In certain embodiments, the disclosure further comprises the use of mouthwash containing appropriate nitrite reducing agents that effectively provide anti-caries activity to reduce dental plaque and gingivitis in humans. Following use of the toothpaste (and/or mouthwash), saliva test strips are used to monitor antimicrobial nitrogen oxides levels generated in the mouth. A nitric oxide generating paste and mouthwash used in context with intra-daily self-monitoring with a saliva test strip for nitrogen oxide promotes consumer compliance behavior. In a further embodiment of the invention, colorimetric test strip outcomes are recorded and tracked on electronic monitoring devices such as wearables (smart watches, activity trackers, wearable cameras, smart glasses, smart clothing, mobile phones etc) providing reminders and updates to both user/patient and dentist or healthcare provider through wireless messaging capabilities as to compliance and adherence to oral healthcare.

In one aspect, the present invention comprises a method for improving oral health in a subject comprising, use of an oral cleanser by a subject, wherein the oral cleanser comprises a potassium nitrate ion source; and further comprises the use of a saliva test strip for measuring nitrite and nitrogen oxide in the oral cavity of the subject. In some aspects, the oral cleanser comprises toothpaste wherein the toothpaste comprises a storage stable, anti-plaque and anti-gingivitis toothpaste having consumer acceptable taste. The toothpaste may comprise 2 to 8% potassium nitrate (by weight based on the total weight of the toothpaste) and may further comprises 0.1-0.3% sodium fluoride. In some embodiments, the toothpaste further comprises salicylic acid, ascorbic acid, or salicylic acid and ascorbic acid and/or nitric oxide donor generators, including but not limited to, S-nitrosothiol, diazeniumdiolate, NONOate, furoxan, nitroaspirin, or inorganic nitrate, such as potassium nitrate.

The present invention comprises a novel approach for the treatment and/or prevention of bacterial, viral, or fungal conditions in the oral cavity and a programmatic approach to monitor it for maximal antimicrobial activity. One component of the program comprises providing a source of nitric oxide precursor(s) within an oral cleanser such as a toothpaste and/or mouthwash, including but not limited to potassium nitrate which may be substituted with nitric oxide donor generators, including, but not limited to, nitrite, S-nitrosothiol, diazeniumdiolate, NONOate, furoxan, nitroaspirin, inorganic nitrate. Though not wishing to be bound by the following theory, it is expected that acidification of the nitric oxide precursors occurs in the sulcus or subgingival space of infected pockets and said source of nitrite ions derived from nitrate precursor are further reduced to form antimicrobial nitrogen oxide, in particular nitric oxide. An aspect of the disclosure pertains to the source of nitrate, optionally reduced by microflora in the oral cavity that can be further reduced in the presence of salicylic acid and/or ascorbic acid which increases the antimicrobial effects within the oral cavity. A further aspect of the disclosure comprises the use of saliva test strips to monitor nitrate wherein to achieve antimicrobial activity, test strips are used to monitor the nitrite in the oral cavity.

Suitable strips for use with the present disclosure comprise a single device, having a three-step method for collecting, transferring, and measuring saliva fluid analytes, specifically for nitrogen oxide anions, nitrite, an analyte of and biomarker for nitric oxide. In one embodiment, the saliva strips, comprise BERKELEY TEST® saliva strips, and they not only monitor nitric oxide status, they also provide an instant readout such that the user can make informed decisions, in real-time, about maintaining a level sufficient of nitrite in the oral cavity to maintain oral health. The BERKELEY TEST® saliva strips enable users (such as those prone to dental problems) who need a sensitive, easy to use, and affordable test that can be used several times daily to make hygiene and dietary adjustments as needed to maintain optimal levels of nitric oxide.

The present invention generally comprises the use of a saliva strip that consists of a single unit comprising an elongated strip, wherein the strip contains a scored mark (or crease) at the midpoint of the strip and wherein the strip contains an absorbent pad at each end. The scored mark enables the strip to be folded easily, thereby, allowing pads at each end of the strip to make contact. The strip contains a first absorbent pad at one end and a second absorbent pad at the opposite end: the first absorbent pad comprises a fluid collection pad, and the second absorbent pad comprises a test pad. The fluid collection pad may comprise a wicking pad, membrane, paper, resin, sponge, immunoabsorbent pad, ionic or other suitable platform that absorbs saliva analytes to be transferred to the test reagent pad, known to those skilled in the art. The test pad enables dry reagent detection chemistry comprising components modified from the Griess diazotization reaction, comprising mixture of naphthylenediamine-dihydrochloride, and sulphanilamide in acidic solution or para-arsanilic acid; and other reactive components known to those skilled in the art. In certain embodiments, the test pad comprises more than one testing zone so that the fluid may be analyzed for more than one biomarker.

In one embodiment at one end of the strip, the fluid collection pad collects the saliva when inserted into the mouth with absorption pad down or in contact with tongue. In some embodiments, this portion of the test strip may optionally include an identifying marking on the strip such that the user can easily identify it as the portion that is to be inserted into the mouth. After absorption of saliva, the strip is removed from the mouth and folded along the scored line or crease to allow the absorption pad to make contact with the test pad, resulting in the transfer of saliva from the absorption pad to the test pad. The test pad, which is not inserted in the mouth, contains dry chemical reagents which contains components modified from the Griess reaction, either naphthylenediamine-HCL and sulphanilamide or para-arsanilic acid, which display a color product upon contact with saliva fluids containing nitric oxide analyte, nitrite; the color product intensity correlates with a concentration of the saliva nitric oxide analyte, nitrite. The Griess reaction and reagents used to detect and measure nitrite is well known to someone skilled in the art. A color scale is provided for the user so that a correlation can be made to designate physiological nitric oxide status. In certain embodiments, the color chart provides a range that assesses nitric oxide levels starting with “depleted” (nitric oxide poor-diet), then “low”, then “threshold” (nitric oxide-rich diet), then “target” and finally “high”. As the color intensity increases, the levels of nitric oxide increase.

The unique design of strip used herein enables easy collection of the test fluid, without requiring an additional vessel, or direct use of fingers to collect or distribute the fluid. In certain embodiments, the absorption pad (fluid collection pad), located on the same side, but opposite end of the elongated strip from that of the test pad, is inserted into the mouth, (under the tongue or sublingually) for a certain period of time within the range of 2-60 seconds, 3-20 seconds or 3-10, most preferably 3-5 seconds, to absorb saliva; the strip is then folded with the thumb and forefinger so that the absorption pad and test reagent pad make contact for 2-60 seconds, 3-20 seconds most preferably 3-5 seconds. Upon release and separation of the absorption pad from the test pad, a colorimetric reaction, based on the chemical detection reagents used, will take place within 45-60 seconds on the test reagent pad resulting in a color intensity and hue that correlates with a physiological concentration of the nitric oxide analyte.

Once the saliva is applied to the test pad for a predetermined amount of time and reaction is allowed to take place therein, the results manifest as a color change. Typically, the original color of the test pad is white, following the chemical reaction between the test fluid and the reagents in the test pad, the color of the test pad changes from white to another color. The resulting color is “matched” with a color scale and wherever the color falls within the scale, a representative physiological nitric oxide content is evaluated. In essence, color intensity on the test pad is compared to a color chart corresponding to physiological concentrations of the nitric oxide analyte.

Alternative embodiments optionally provide in addition to a nitric oxide status detection pad, multiple, separate test pads, each containing dry chemical reagents responsive to different concentrations of nitric oxide analyte attached to the strip, thereby, increasing the sensitivity; here, the absorption pad is enlarged to cover multiple, separate pads.

In an embodiment, the present disclosure provides a method for improving oral health in a subject comprising, using a vehicle for increasing the presence of nitrate in the oral cavity of a subject; monitoring the presence of nitrate to nitrite bioconversion in the oral cavity of a subject using a saliva test strip for measuring nitrite in the oral cavity of the subject, and implementing oral hygiene steps when the saliva test strip indicates certain levels of nitrite metabolites. In an embodiment, the method encompasses the use of a vehicle for increasing the presence of nitrate comprises the introduction of a nitrate-based substance into the oral cavity. In an embodiment, the nitrate-based substance comprises a gum or a lozenge, the gum may comprise an inorganic nitrate donor and a gum base, and may further comprise flavorings, binders, fillers, bicarbonate or Vitamin C. The inorganic nitrate donor of the gum may comprise potassium nitrate, and the gum base may comprise natural gum base, chicle, synthetic polymers, plasticizers, or resin, further comprising flavorings (natural flavorings, artificial flavorings, sugar sweeteners, non-sugar sweeteners). As disclosed herein the vehicle for increasing the presence of nitrate may comprise an oral cleanser, wherein the oral cleanser comprises toothpaste, mouthwash, or dental strips, in an embodiment, the toothpaste comprises a storage stable, anti-plaque and anti-gingivitis toothpaste having consumer acceptable taste. In certain embodiments, the toothpaste comprises 2 to 8% potassium nitrate by weight based on the total weight of the toothpaste, the toothpaste further comprises 0.1-0.3% sodium fluoride and the toothpaste further comprises salicylic acid, ascorbic acid, or salicylic acid and ascorbic acid.

The methods of the present disclosure comprise the use of a saliva test strip comprising an elongated strip, wherein the strip contains a scored mark at the midpoint of the strip and wherein the strip contains a first absorbent pad at one end and a second absorbent pad at the opposite end, wherein the first absorbent pad comprises a fluid collection pad, and wherein the second absorbent pad comprises a test pad, and wherein the test pad enables dry reagent detection chemistry comprising components modified from the Griess diazotization reaction, comprising mixture of N-naphthylenediamine-dihydrochloride and sulphanilamide, or N-(naphthyl)ethylenediammonium-dihydrochloride and sulphanilamide, or para-arsanilic acid N-ethylenediamine tetrahydroquinoline, or para-arsanilic acid N-(1-naphthyl)ethylenediamine dihydrochloride; wherein the fluid collection pad is inserted into the mouth for a first predetermined period of time to absorb saliva, wherein the device is then folded so that the fluid collection pad and test pad make contact for a second predetermined period of time, wherein the contact between the fluid collection pad and test pad results in a color change on the test pad following a third predetermined period, wherein the color change is compared to a color scale and wherever the color falls within the scale, nitric oxide content is evaluated. Certain levels of nitrate to nitrite bioconversion are indicated by the color scale consisting of 0 to 1,000 uM nitrite.

In an embodiment, the methods of the disclosure comprise oral hygiene steps including, but not limited to, chewing a nitrate-based gum, brushing teeth, using mouthwash, flossing, or cleaning the oral cavity. The methods relate to improving oral health including but not limited to, reduction of dental plaque, gingivitis, oral infections, tooth decay, tooth loss, gum disease, oral cancer, throat cancer, oral sores, periodontal disease, gum disease, dental cavities, gingival recession and halitosis.

In an embodiment, the colorimetric reading of the strip is recorded by a wearable which may serve as a spectrometer to track and provide reminders and updates to both user and dentist or healthcare provider through wireless messaging capabilities. In an embodiment, the wearable comprises a smart watch, activity tracker, wearable camera, smart glass, smart clothing, or mobile phone.

Disclosed herein is a gum composition comprising an inorganic nitrate donor and a gum base, wherein the inorganic nitrate donor comprises potassium nitrate, wherein the gum base comprises natural gum base, chicle, synthetic polymers, plasticizers, or resin. The gum composition may further comprise natural flavorings, artificial flavorings, sugar sweeteners, non-sugar sweeteners, food coloring, binders, fillers, bicarbonate or Vitamin C. In an embodiment, the inorganic nitrate donor comprises potassium nitrate, wherein the potassium nitrate is present in the amount of 2-400 mg per unit of gum. In an embodiment, the inorganic nitrate donor comprises a plant-based bioequivalent of nitrate at 2-400 mg per unit wherein the nitrate donor is derived from spinach, kale, beet, celery, arugula, fennel and/or combinations thereof, nitrate-rich plant-derived powders, or nitrate-rich juice concentrate. In an embodiment, the gum base may comprise chicle, synthetic polymers, plasticizers, or resin, the flavorings comprise natural flavorings, artificial flavorings, sugar sweeteners, non-sugar sweeteners, fillers optionally included may include magnesium stearate, or silicon dioxide and the like. In an embodiment, the sugar sweeteners include but are not limited to sugar alcohols, maltitol, sorbitol, isomalate, sucralose, and the non-sugar sweeteners include, but are not limited, to xylitol. In an embodiment, the flavorings may comprise spearmint, orange, apple, strawberry, cinnamon, cloves, fennel or blueberry.

In an embodiment, the gum composition comprises potassium nitrate at 2-400 mg per unit with the remainder of 0.5-5 gm consisting of a gum base, maltitol, sorbitol, isomalate, natural and artificial flavors, vegetarian magnesium stearate, sucralose, silicon dioxide with bicarbonate, and vitamin C.

In an embodiment, the gum composition comprises potassium nitrate at 2-400 mg per unit with the remainder of 0.5-5 gm consisting of a gum base, maltitol, sorbitol, isomalate, natural and artificial flavors, vegetarian magnesium stearate, sucralose, and silicon dioxide.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific effective amount for any particular subject will depend upon a variety of factors including the age, body weight, general health, sex and diet of the patient; the time of administration; the route of administration; and like factors well known in the field of health.

Disclosed are the components to be used to prepare compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of ingredients A, B, and C are disclosed as well as a class of ingredients D, E, and F and an example of a combination substance, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, “gum” means a soft, chewable, cohesive substance designed to be chewed without being swallowed. In an embodiment, the gum may be composed of gum base, sweeteners, softeners/plasticizers, flavors, colors, and, optionally a hard or powdered polyol coating. Its texture may be reminiscent of rubber because of the physical-chemical properties of its polymer, plasticizer, and resin components, which contribute to its elastic-plastic, sticky, chewy characteristics.

As used herein, toothpaste means a paste or substance that is used to clean teeth, maintain oral hygiene and improve the aesthetic of the teeth. In certain embodiments, toothpaste may comprise abrasives, fluorides, surfactants, antibacterial agents, flavorants, remineralizers, whiteners and the like.

Mouthwash means a liquid which is held in the mouth passively or swilled around the mouth by contraction of the perioral muscles and/or movement of the head, and may be gargled where the head is tilted back and the liquid bubbled at the back of the mouth. In an embodiment, mouthwash may be antiseptic, analgesic, anti-inflammatory, or anti-fungal. In an embodiment, mouthwash may contain a variety of ingredients including but not limited to alcohol, antibiotics, fluoride, flavoring agents, betamethasone, calcium, methyl salicylate, triclosan, water, zinc and the like.

Dental strip a strip or tape comprising a substance, such as a whitener or other component that may improve oral aesthetics and/or oral hygiene.

Oral hygiene includes, but is not limited to the practice of keeping the mouth and teeth clean to prevent dental problems, most commonly, dental cavities, gingivitis, periodontal (gum) diseases, bad breath, periodontitis, and dental trauma, subluxation, oral cysts. In an embodiment, processes for maintaining oral hygiene include removing plaque, flossing, interdental brushing, tongue scraping, oral irrigation and the like.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

The following specific examples will illustrate the invention as it applies to the methods of improving oral health by detecting and monitoring biomarkers such as nitrites in saliva. It will be appreciated that other examples, including minor variations in procedures will be apparent to those skilled in the art, and that the invention is not limited to these specific illustrated Examples.

### EXAMPLES

### Example 1

### Bioconversion of Nitrate Following the Use of Nitrate-Rich Gum

As discussed herein, dietary inorganic nitrate (NO3) and its reduced forms nitrite (NO2) and nitric oxide (NO), respectively, are of critical importance for host defense in the oral cavity. High concentrations of salivary nitrate are linked to a lower prevalence of caries due to growth inhibition of cariogenic bacteria. Anti-carogenic activity is dependent upon the bioconversion of NO3 to NO2 and downstream nitrogen oxides, specifically, antimicrobial nitric oxide.

This example demonstrates a programmatic approach for providing a source of dietary nitrate and methods to ensure bioconversion is taking place within the mouth, as well as a means to sustain antimicrobial levels. Although it is well established that nitrogen oxide may exhibit antimicrobial activity, at present no method or invention ensures the validation of the nitrate source and whether the body is bio-converting nitrate to a downstream metabolite that exhibits antimicrobial activity.

The current example demonstrates an end-to-end solution for implementing, maintaining and improving oral hygiene: 1. dietary nitrate (NO3) source in a delivery format that optimizes immediate local levels of nitrate, including but not limited to nitrate-rich gum, lozenges, paste, mouthwash and the like. 2. saliva test strips to both ensure, indirectly, NO3 content and validate bioconversion of nitrate to antimicrobial nitrite 3. a method to track and record bioconversion of nitrate to nitrite in context of a dietary nitrate delivery format, such as nitrate-rich gum.

### Bioconversion of Nitrate Following the Use of Nitrate-Rich Gum

Prior to the introduction of a nitrate rich source, the oral cavity of a subject was depleted of bio-converted nitrate (22 uM) as measured by BERKELEY TEST® saliva test strips. Upon chewing a nitrate-rich gum of >150 mg per piece, the strips detected the presence of nitrite within 20 min (109 uM). In this example, the levels of nitrite rose over a 4-hour period to a target level of 326 uM nitrite.

Table 1 below provides a representation showing a rise of bio-converted nitrate to nitrite in the mouth upon chewing the potassium nitrate containing gum.

Within a few minutes, nitrate is converted to nitrite and the activity persists for up to 2-4 hours within the mouth even after the gum is removed within a 30 min to 2 hours time period.

TABLE 1

| | Nitrite (NO2) | |
|---|---|---|
| Time | Concentration | Activity |
| 7:36 am | 22 uM | Test |
| 7:37 am | Introduced Nitrate | Began |
| | (NO3) gum | chewing |
| 7:55 am | 109 | Test |
| 8:21 am | 163 | Test |
| 8:39 am | 217 | Test |
| 8:53 am | 217 | Test |
| 9:06 am | 326 | Test |
| 10:10 am | 326 | Test |

### Example 2

### Bioconversion of Dietary Nitrate to Nitrite in the Mouth Reduces the Acidification of the Mouth: Salivary Nitrate Bioconversion Correlates with Elevating pH

The present example demonstrates that the formation of antimicrobial nitric oxide results in an increase of the pH preventing erosion of tooth enamel.

Here in a clinical study with 46 subjects, NO3-rich dietary source exhibited a protective effect against caries by an increase of salivary pH as shown in the FIGS. 8 and 9. The protocol is detailed in the publication, Sustaining elevated levels nitrite in the oral cavity through consumption of nitrate-rich beetroot juice in young healthy adults reduces salivary pH, Hohensinn, B., et al. (2016) Nitric Oxide 60: 10-15.

The results (FIG. 8) show that, in comparison to a placebo group (open circles), consumption of nitrate-rich juice (closed circles) resulted in the bioconversion of nitrate to nitrite, as determined by the saliva test strips. With the bioconversion of nitrate to nitrite, the pH of saliva rose from 7.0 to 7.5, confirming the anti-cariogenic effect of provide a dietary NO3 source (see FIG. 9).

These results clearly indicate that NO3-rich dietary source significantly prevents the acidification of the salivary pH if consumed regularly and bioconversion of NO3 to NO2 occurs as determined by a saliva NO test strip.

As to specific, salivary nitrite levels, a surrogate of nitric oxide, were determined by using BERKELEY TEST® saliva test according to the following instructions.
The test strips exhibit a saliva absorption pad on one end, and a NO test pad on the other end. First, the saliva collection pad was placed for 5 seconds on the tongue.
The strip was removed and the two ends were gently pressed against each other for 5 seconds.
After releasing the test pad the developing color was observed and compared to the color chart illustrated on the packaging of the test strips.
A dark pink color indicated a high NO level, whereas light pink was linked to NO depletion. BERKELEY TEST® saliva test strips are based on the well-established, modified Griess reagent reaction, which is often used in evaluating serum, urine, and saliva NO2.

The strips do not measure NO3. Hence, the bioconversion of NO3 to NO2, which occurs in the mouth via the nitrate-reducing microflora, is detected by the strips. If bioconversion does not occur, the strips do not display a reaction. BERKELEY TEST® saliva test trips are calibrated as: depleted, low, threshold, target, and high, which translates to approximately 21, 108, 217, 434, and 869 μM NO2, respectively. The pH of all centrifuged saliva samples was measured using a pH meter. As determined by salivary NO test strips, which is reflective of total bioavailability of NO and assesses the bioconversions of NO3 to NO2—in the mouth, a required and necessary step in the formation of NO.

Within the clinical assessment peak levels of 100-200 μM (level 2-3 in FIG. 10) were detected with the test strips within the first 2 hours, which approximates the time for bioconversion to take place. Basal mean levels of salivary NO after an overnight fast were found to be less than 30 μM, which is consistent with the chewing gum in FIG. 8). Two hours after dietary NO3 the saliva levels increased to 100-200 μM, which was significantly higher than NO levels of the placebo group (30 μM). In the evening NO levels of the NO3 source declined, but were still significantly higher than NO levels of the placebo group.

Thus, dietary NO3 needs to be constantly ingested in order to benefit from the antimicrobial effects and other biological functions of NO. Hence, the importance of intra-daily monitoring with salivary nitric oxide test strips.

It would not be unreasonable to predict that if oral bioconversion levels were sustained throughout the day, the mouth would remain alkaline, as reflected by a cytocidal effect of the acid-forming bacteria. In this case, the strips would be used to remind the patient to replenish with NO3-rich foods and the like, including but not limited to gum, lozenges, paste, and/or mouthwashes, as the levels of detected nitrites begin to naturally fall through the day. In doing so, salivary pH values would seldom become acidic and harmful to the teeth.

pH is a prominent indicator for the presence and number of cariogenic bacteria, and elevated pH values result from a decline in the number of acid-producing bacteria. Thus, to shift from a transient cytostatic to a lasting cytocidal effect on cariogenic bacteria, dietary NO3-foods etc should be incorporated into one's oral health program as regulated by intra-daily readings with a salivary NO test strips. Individuals will vary as to how long their NO3-/NO2-/NO levels are maintained, hence, it will be critical to monitor frequently with test strips and, if levels are falling, replenish with NO3 local oral source before acidogenic bacteria reestablish.

An important aspect in this regard is the efficacy of commercial antibacterial mouthwash products in comparison to a NO3 source. Non-NO3 washes products reduce the growth of the entire NO3-reducing oral microbiome, ie, nitrate-reducing bacteria and thus inhibit the bioactivation of NO3 and all its health beneficial functions. Hence, NO3 foods, gums, and oral pastes and washes which may lead to a selective cytostatic or cytocidal effect of acidogenic bacteria assuming antimicrobial levels are sustained as determined by test strips.

This invention demonstrates that nitrate-rich source holds potential effects against dental caries via bioconversion of NO3 to NO2 resulting in elevating pH levels which is predictably a reduction of acidogenic cariogenic bacteria. However, persistent anti-cariogenic effect which is likely due to the clearance of NO3/NO2 which needs to be periodically replenished based on test strip outcomes.

### Example 3

### Nitrate-Rich Gum

A specific composition of the nitrate-rich gum of the present disclosure is provided in Table 2 below. In certain embodiments, the nitric oxide donor is KNO3, in other embodiments, the KNO3 may be substituted by other nitrate/nitrite donors, including but not limited to NONOate, nitrite, or plant-derived powder from leafy greens or beets rich in nitrate. In certain embodiments, the gum base comprises a natural gum base, chicle, or synthetic gum bases, including, but not limited to, resin, waxes and elastomer. In certain embodiments, sweeteners used herein are known to those skilled in the art and include, but are not limited to natural or synthetic sweeteners including maltitol, sorbitol, sucralose, isomalate, xylitol, stevia and the like. In an embodiment, the plasticizer may comprise vegetable magnesium stearate and silicon dioxide, the flavorings may be natural or artificial and comprise spearmint, orange, apple, strawberry, cinnamon, cloves, fennel or blueberry, additives may include vitamins, such as Vitamin C, and the coating may comprise sorbitol, maltitol, and isomalate.

In an embodiment, NO saliva test strips, such as BERKELEY TEST® saliva strips, are provided with each piece of gum or serving to enable assessment of nitrate to nitrite bioconversion, i.e., test before and after chewing gum.

TABLE 2

| Ingredient | % by Weight Composition |
|---|---|
| KNO3 | 5-10% |
| Gum Base | 25-35% |
| Sweetener | 40% |
| Sugar Alcohol | <1% |
| Plasticizer | 1-2% |
| Flavors | 1-3% |
| Additives | 1-5% |
| Coating | Based on Size |

While the present disclosure has been discussed in terms of certain embodiments, it should be appreciated that the present disclosure is not so limited. The embodiments are explained herein by way of example, and there are numerous modifications, variations and other embodiments that can be employed that would still be within the scope of the present disclosure.

1. A method for improving oral health in a subject comprising,
using a vehicle for increasing the presence of nitrate in the oral cavity of a subject;
monitoring the presence of nitrate-to-nitrite bioconversion in the oral cavity of a subject using a saliva test strip for measuring nitrite in the oral cavity of the subject, and
implementing oral hygiene steps when the saliva test strip indicates detects nitrate-to-nitrite bioconversion.

2. The method of claim 1, wherein the vehicle for increasing the presence of nitrate comprises the introduction of a nitrate-based substance into the oral cavity.

3. The method claim 2, wherein the nitrate-based substance comprises a gum or a lozenge.

4. The method of claim 3, wherein the gum comprises inorganic nitrate potassium nitrate, nitric oxide donors, acidified nitrite, nitrite, organic nitrate, sodium nitroprusside, NONOates, S-nitrosthiols, or probiotic NO producing nanoparticles or substrates contained within a gum base.

5. The method of claim 4, further comprising flavorings, binders, fillers, bicarbonate or Vitamin C.

6. The method of claim 4, wherein the inorganic nitrate donor comprises potassium nitrate,
wherein the gum base comprises natural gum base, chicle, synthetic gum base, polymers, plasticizers, or resin,
wherein the flavorings comprise natural flavorings, artificial flavorings, sugar sweeteners, non-sugar sweeteners.

7. The method of claim 1, wherein the vehicle for increasing the presence of nitrate comprises an oral cleanser, and wherein the oral cleanser comprises toothpaste, mouthwash, or dental strips.

8. The method of claim 7, wherein the toothpaste comprises a storage stable, anti-plaque and anti-gingivitis toothpaste having consumer acceptable taste.

9. The method of claim 1, wherein the toothpaste comprises 2 to 8% potassium nitrate by weight based on the total weight of the toothpaste, wherein the toothpaste further comprises 0.1 to 0.3% sodium fluoride and wherein the toothpaste further comprises salicylic acid, ascorbic acid, or salicylic acid and ascorbic acid.

10. The method of claim 1, wherein the saliva test strip comprises an elongated strip, wherein the strip contains a scored mark at the midpoint of the strip and wherein the strip contains a first absorbent pad at one end and a second absorbent pad at the opposite end, wherein the first absorbent pad comprises a fluid collection pad, and wherein the second absorbent pad comprises a test pad, and wherein the test pad enables dry reagent detection chemistry comprising components modified from the Griess diazotization reaction, comprising mixture of N-naphthylenediamine-dihydrochloride and sulphanilamide, or N-(naphthyl)ethylenediammonium-dihydrochloride and sulphanilamide, or para-arsanilic acid N-ethylenediamine tetrahydroquinoline, or para-arsanilic acid N-(1-naphthyl)ethylenediamine dihydrochloride; wherein the fluid collection pad is inserted into the mouth for a first predetermined period of time to absorb saliva, wherein the device is then folded so that the fluid collection pad and test pad make contact for a second predetermined period of time, wherein the contact between the fluid collection pad and test pad results in a color change on the test pad following a third predetermined period, wherein the color change is compared to a color scale and wherever the color falls within the scale, nitric oxide content is evaluated.

11. The method of claim 10, wherein nitrate to nitrite bioconversion is indicated by the color scale consisting of 0 to 1,000 uM nitrite.

12. The method of claim 1, wherein implementing oral hygiene steps comprises chewing a nitrate-based gum, brushing teeth, using mouthwash, flossing, or cleaning the oral cavity.

13. The method of claim 1, wherein improving oral health comprises reduction of dental plaque, gingivitis, oral infections, tooth decay, tooth loss, gum disease, oral cancer, throat cancer, oral sores, periodontal disease, gum disease, dental cavities, gingival recession and halitosis.

14. A gum composition comprising nitric oxide donor contained within a gum base,
wherein the nitric oxide donor comprises potassium nitrate, acidified nitrite, sodium nitrite, organic nitrates, sodium nitroprusside, NONOates, S-nitrosthiols, or NO producing probiotics encapsulated in nanoparticles, and
wherein the gum base comprises natural gum base, chicle, or a synthetic gum, including polymers, plasticizers, or resin.

15. The gum composition of claim 14, further comprising natural flavorings, artificial flavorings, sugar sweeteners, non-sugar sweeteners, food coloring, binders, fillers, bicarbonate or Vitamin C.

16. The gum composition of claim 14, wherein the inorganic nitrate donor comprises potassium nitrate, and wherein the potassium nitrate is present in the amount of 2-400 mg per unit of gum.

17. The gum composition of claim 16,
wherein the gum base comprises, chicle or synthetic polymers, plasticizers, or resin,
wherein the flavorings comprise natural flavorings, artificial flavorings, sugar sweeteners, non-sugar sweeteners,
wherein the fillers comprise magnesium stearate, isomalate, or silicon dioxide.

18. The gum composition of claim 17, wherein the sugar sweeteners comprise sugar alcohols, maltitol, sorbitol, sucralose, wherein the non-sugar sweeteners comprise xylitol, wherein the flavorings comprise spearmint, orange, apple, strawberry, cinnamon, cloves, fennel or blueberry.

19. The gum composition of claim 16, wherein the inorganic nitrate source comprises a plant-based bioequivalent of nitrate at 2-400 mg per unit gum and wherein the nitrate source is derived from spinach, kale, beet, celery, arugula, fennel or combinations thereof, nitrate-rich plant-derived powders, or nitrate-rich juice concentrate contained within a natural or synthetic chewing gum base admixed with sugar or non-sugar sweeteners, fillers, binders, and natural or artificial flavors.

20. The gum composition of claim 14 and method of claim 10, wherein the gum is co-bundled or packaged with saliva nitric oxide strips to test for nitrate-to-nitrite bioconversion within the oral cavity and to monitor the persistence of bioconversion during and after chewing nitrate-containing gum.
